(19) United States
(12) Patent Application Publication
(10) Pub. No.: US 2012/0041025 A1
(43) Pub. Date: Feb. 16, 2012

(54) Dihydrolipoic Acid Derivatives Comprising Nitric Oxide and Therapeutic Uses Thereof

(76) Inventor: Sampath Parthasarathy, Tucker, GA (US)

(21) Appl. No.: 13/201,705

(22) PCT Filed: Feb. 19, 2010
(86) PCT No.: PCT/US10/24772

§ 371 Date: Oct. 27, 2011

Related U.S. Application Data
(60) Provisional application No. 61/207,781, filed on Feb. 17, 2009.

(51) Int. Cl. A61K 31/445; A61K 31/40; C07C 381/00; A61K 31/201; A61K 31/23; C07D 211/60; C07D 317/64; A61K 31/36; A61P 9/10; A61P 3/00; A61P 3/10; A61P 13/12; A61P 3/06; A61P 29/00; A61P 9/08; C07D 207/16

(52) U.S. Cl. 514/315; 548/533; 514/423; 554/85; 514/560; 514/549; 546/245; 549/437; 514/466

(57) ABSTRACT

Compounds are provided that comprise dinitroso-derivatives of dihydrolipoic acid. Pharmaceutical compositions comprising the compounds and methods of using the compounds for treating various diseases and disorders, including angina, hypertension, diabetes, dyslipidemia, renal insufficiency, myocardial infarction, stroke, atherosclerosis, and the target organ damage that accompanies these various diseases and disorders, are further provided. The compounds are useful in improving vasodilation, reducing low-density lipoprotein oxidation, and reducing inflammation in a subject.

### CROSS-REFERENCE TO RELATED APPLICATIONS

The present application claims the benefit of U.S. Provisional Application Ser. No. 61/207,781, filed Feb. 19, 2009, the entire disclosure of which is incorporated herein by reference.

### FIELD OF THE INVENTION

The present invention relates to compounds comprising nitric oxide derivatives of dihydrolipoic acid (DHLA) and methods of using the same. In particular, the present invention relates to S-nitrosothiol compounds that are derived from DHLA and are useful in the treatment of a number of diseases and disorders, including angina, hypertension, diabetes, dyslipidemia, renal insufficiency, myocardial infarction, stroke, atherosclerosis, and target organ damage that accompanies these various diseases and disorders. In addition, the present invention relates to the use of compounds comprising nitric oxide derivatives of DHLA in improving vasodilation, reducing low-density lipoprotein oxidation, reducing renal insufficiency, and reducing inflammation in subjects in need of such treatment.

### BACKGROUND OF THE INVENTION

Currently, a number of anti-inflammatory agents and antioxidants are available, or are naturally-occurring, and are capable of reducing the amount of oxidative stress or inflammation in patients. In plants and animals, one such agent is alpha lipoic acid. Alpha lipoic acid, also known as thioctic acid, is a naturally-occurring 8-carbon fatty acid that is synthesized by plants and animals, including humans, and serves several important functions in the body. Alpha lipoic acid contains two sulfur atoms that are normally found in an oxidized, disulphide form, but which can be reduced to form thiols and form dihydrolipoic acid (DHLA). Indeed, the body of an individual routinely converts some alpha lipoic acid to DHLA, and it is believed that DHLA may function as a more powerful antioxidant when compared to alpha lipoic acid. In this regard, it has also been observed that free alpha lipoic acid is rapidly taken up by cells and is reduced, intracellularly, to DHLA, which can then be rapidly secreted from cells and act as a potent antioxidant along with alpha lipoic acid.

As potent antioxidants, alpha lipoic acid and DHLA are able to scavenge various free radicals and oxidants including hydroxyl radicals, singlet oxygens, peroxynitrite, and hypochlorous acid. Because these free radicals have been implicated in the pathophysiology of many chronic diseases, it is believed that the pharmacotherapeutic effects of the various forms of lipoic acid are largely due to its antioxidant properties. In addition to its antioxidant properties, however, lipoic acid is also a potent anti-inflammatory reagent. Lipoic acid inhibits the activation of IKK/NF-κB signaling which plays a central role in inflammatory responses. Furthermore, numerous other health benefits have been attributed to lipoic acid including lowering cholesterol, increasing glucose uptake by cells, stimulating neurological function, decreasing liver toxicity, increasing levels of glutathione and ascorbic acid, and preventing stroke. Additionally, a recent report has also demonstrated that alpha lipoic acid inhibits atherosclerotic lesion development, due at least in part to its anti-inflammatory effect (Zhang W, et al. Dietary α-Lipoic Acid Supplementation Inhibits Atherosclerotic Lesion Development in Apolipoprotein E-Deficient and Apolipoprotein E/Low-Density Lipoprotein Receptor-Deficient Mice. Circulation. 2008; 117: 421-428).

Another molecule with great pharmaceutical potential is nitric oxide (NO). As would be commonly recognized, nitroglycerine is often prescribed to reduce the pain of angina and does so by generating NO, which relaxes the walls of the coronary arteries and arterioles. However, NO has also been shown to be a highly potent regulatory molecule that mediates a variety of other physiological effects. For example, NO is capable of governing blood pressure, dilating blood vessels, and controlling the action of almost every muscle in an individual. The immune system also uses NO in combating viral, bacterial, and parasitic infections, and it has further been shown that the immune system utilizes NO in combating tumors. Additionally, NO transmits messages between nerve cells, and has thus been implicated with the processes of learning, memory, sleep, pain, and depression.

Nevertheless, although certain health benefits have been attributed to the administration of lipoic acid and NO-donor molecules, such as nitroglycerine, lipoic acid still continues to be largely viewed as only a nutraceutical supplement and many NO-donor molecules continue to be used for only specific applications. However, the fact that NO can react with thiols to form S-nitrosothiols, such as S-nitrosocysteine and S-nitrosoglutathione, in vivo and form important NO-donor molecules is beginning to be elucidated. As such, many S-nitrosothiols are now being synthesized chemically and are showing promise as clinically-useful NO-donor drugs (See, e.g., Miller M R, et al. Recent Developments in Nitric Oxide Donor Drugs. Brit. J. Pharm. 151: 305-321 (2007), which is incorporated herein by reference).

To date, however, these S-nitrosothiol compounds have proven to be unstable at room temperature or when exposed to light and, as such, the full extent of the underlying health benefits that may be obtained with NO-donor molecules have yet to be fully realized. Additionally, it remains unknown as to how an NO group can be stably and effectively incorporated into the structure of a lipoic acid compound, such as DHLA, so that a compound could be designed to obtain the maximum benefits associated with a lipoic acid yet also be capable of serving as a stable and effective NO-donor molecule. Indeed, to date, a sufficient lipoic acid compound has failed to be combined with an NO group, such that the beneficial properties of lipoic acid and those of NO could be combined into one compound that is capable of exhibiting a variety of multi-functional therapeutic effects by targeting multiple diseases and disorders, and their underlying causes, with minimal toxicity.

Accordingly, a compound that combined a lipoic acid compound with an NO group would be highly desirable and potentially very beneficial in treating a variety of diseases and disorders.

### SUMMARY OF THE INVENTION

It is thus an object of the present invention to provide compounds that comprise nitric oxide derivatives of dihydrolipoic acid (DHLA), which can provide the beneficial properties of DHLA and yet not interfere with the ability of the compounds to serve as an effective NO-donor and thus be utilized in methods of treating a variety of diseases and disorders where the actions of DHLA and NO are indicated.

It is also an object of the present invention to provide methods of treating various diseases and disorders, including angina, hypertension, diabetes, dyslipidemia, renal insufficiency, myocardial infarction, stroke, atherosclerosis, and the target organ damage that accompanies these various diseases and disorders, by administering an effective amount of a compound of the present invention to a subject in need of treatment.

It is another object of the present invention to provide methods for improving vasodilation wherein a subject in need of such treatment is administered an effective amount of a compound of the present invention to thereby improve vasodilation.

It is yet another object of the present invention to provide methods for reducing low-density lipoprotein oxidation wherein a subject in need of such a reduction is administered an effective amount of a compound of the present invention to thereby reduce a level of low-density lipoprotein oxidation.

It is a further object of the present invention to provide a method of reducing inflammation in a subject in need thereof by administering an effective amount of a compound of the present invention to thereby reduce the amount of inflammation in a subject.

These and other objects are provided by virtue of the present invention which comprises compounds that include beneficial properties of DHLA with those of NO donors. In a preferred embodiment of the present invention, compounds are provided having the following general Formula (I), or a pharmaceutically-acceptable salt or solvate thereof, as follows:

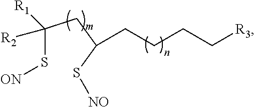

wherein:

m is an integer from 1 to 2;

n is an integer from 1 to 10;

R₁ and R₂ are independently selected from the group consisting of H, methyl, ethyl, propyl, butyl, isopropyl, isobutyl, and tert-butyl; and

R₃ is selected from the group consisting of COOH, COOCH₃, COOCH₂CH₃,

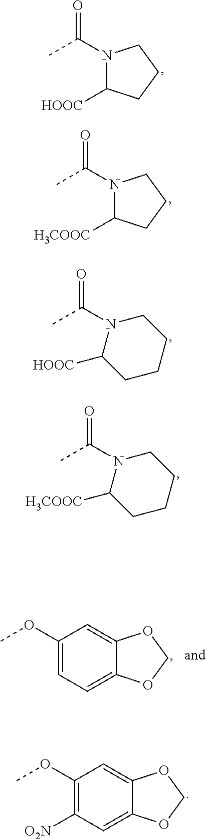

In another preferred embodiment of the present invention, compounds are provided having the general Formula (XV), or a pharmaceutically-acceptable salt or solvate thereof, as follows:

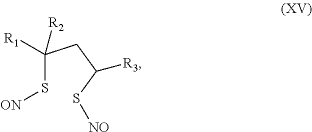

wherein:

R₁ and R₂ are independently selected from the group consisting of H, CH₃, and tert-butyl; and

R₃ is selected from the group consisting of CH₂CHCHCH₂COOCH₃, CH₂CHCHCHCHCOOH, and CHCHCHCHCOOCH₂CH₃.

In addition, the present invention provides pharmaceutical compositions wherein the compounds of the present invention further comprise a pharmaceutically-acceptable vehicle, carrier, or excipient, or are in a sustained-release formulation.

These embodiments and other alternatives and modifications within the spirit and scope of the presently-disclosed invention will become readily apparent to those of ordinary skill in the art after a study of the description, Figures, and non-limiting Examples in this document.

### BRIEF DESCRIPTION OF THE DRAWING FIGURES

FIG. 1 is a schematic representation of the chemical structures of alpha lipoic acid, dihydrolipoic acid (DHLA), a mononitrosolipoic acid (6-mercapto-8-[(oxidoazanylidyne)-λ⁴-sulfanyl]octanoic acid), and a dinitrosolipoic acid (6,8-bis[(oxidoazanylidyne)-λ⁴-sulfanyl]octanoic acid);

FIG. 2 is an image of a western blot for nitrosotyrosine residues subsequent to incubating bovine serum albumin with a dinitroso-derivative of DHLA;

FIG. 3 is a graph showing the visible spectrum of a dinitroso-derivative of DHLA in ethyl alcohol; and

FIG. 4 is a graph showing the inhibition of low-density lipoprotein (LDL) oxidation by contacting the LDLs with various concentrations of a dinitroso-derivative of DHLA.

### DETAILED DESCRIPTION OF THE PREFERRED EMBODIMENTS

In accordance with the present invention, compounds that comprise nitric oxide (NO) derivatives of dihydrolipoic acid (DHLA) are provided. In particular, the present invention provides compounds that include the beneficial properties of DHLA, yet are still capable of serving as stable and effective NO donors. These compounds are useful in treating a variety of diseases and disorders, including angina, hypertension, diabetes, dyslipidemia, renal insufficiency, myocardial infarction, stroke, atherosclerosis, and the target organ damage that accompanies these various diseases and disorders. In particular, in some embodiments, the compounds can be administered to a subject to improve vasodilation, reduce low-density lipoprotein (LDL) oxidation, improve renal insufficiency, or reduce inflammation in a subject in need of such treatment.

In one of the preferred embodiments of the present invention, compounds useful in the invention have the general Formula (I) as follows:

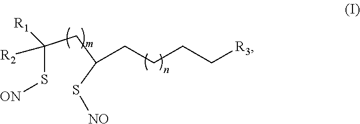

wherein:

m is an integer from 1 to 2;

n is an integer from 1 to 10;

R₁ and R₂ are independently selected from the group consisting of H, methyl, ethyl, propyl, butyl, isopropyl, isobutyl, and tert-butyl; and

R₃ is selected from the group consisting of COOH, COOCH₃, COOCH₂CH₃,

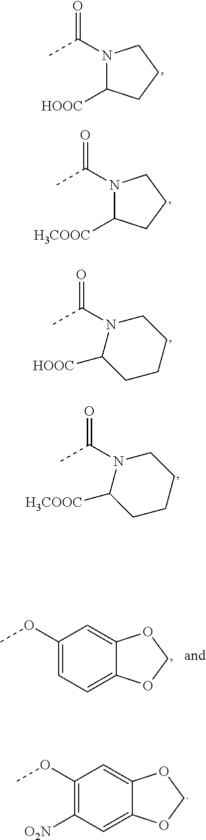

where the dashed bonds (- - -) indicate the point of attachment of the R₃ group to the remainder of the compound.

In one preferred embodiment of the invention, a compound of Formula (I) is provided where m equals 1, n equals 1, R₁ is methyl, R₂ is methyl, and R₃ is COOH, as shown by the following Formula (II):

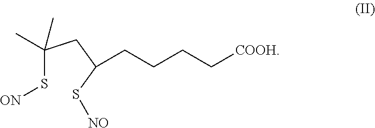

In another preferred embodiment of the invention, a compound of Formula (I) is provided where m equals 1, n equals 1, R₁ is H, R₂ is tert-butyl, and R₃ is COOH, as shown by the following Formula (III):

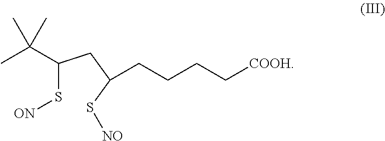

In yet another preferred embodiment of the invention, a compound of Formula (I) is provided where m equals 2, n equals 1, R₁ is methyl, R₂ is ethyl, and R₃ is COOH, as shown by the following Formula (IV):

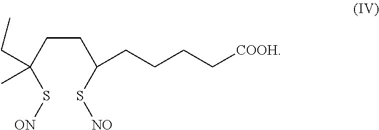

In still another preferred embodiment of the invention, a compound of Formula (I) is provided where m equals 2, n equals 1, R₁ is H, R₂ is tert-butyl, and R₃ is COOCH₂CH₃, as shown by the following Formula (V):

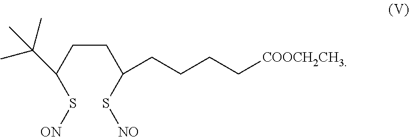

In other preferred embodiments of the invention, a compound of Formula (I) is provided where m equals 1, n equals 1, R₁ is methyl, R₂ is methyl, and R₃ is COOCH₃, as shown by the following Formula (VI):

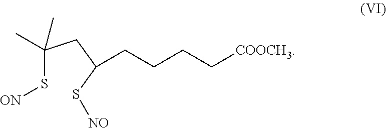

In another embodiment of the invention, a compound of Formula (I) is provided where m equals 1, n equals 5, R₁ is methyl, R₂ is methyl, and R₃ is COOH, as shown by the following Formula (VII):

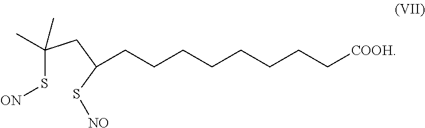

In other embodiments of the invention, a compound of Formula (I) is provided where m equals 2, n equals 4, R₁ is H, R₂ is tert-butyl, and R₃ is COOH, as shown by the following Formula (VIII):

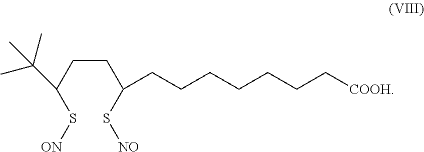

In another preferred embodiment of the invention, a compound of Formula (I) is provided where m equals 1, n equals 1, R₁ is H, R₂ is H, and R₃ is

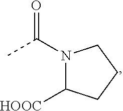

as shown by the following Formula (IX):

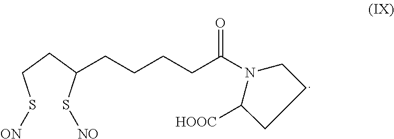

In yet another preferred embodiment of the invention, a compound of Formula (I) is provided where m equals 1, n equals 1, R₁ is H, R₂ is H, and R₃ is

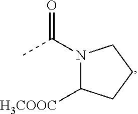

as shown by the following Formula (X):

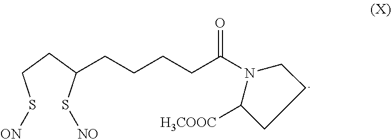

In still another preferred embodiment of the invention, a compound of Formula (I) is provided where m equals 1, n equals 1, R₁ is H, R₂ is H, and R₃ is

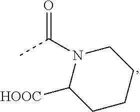

as shown by the following Formula (XI):

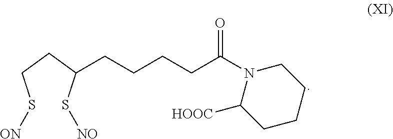

In other preferred embodiments of the invention, a compound of Formula (I) is provided where m equals 1, n equals 1, R₁ is H, R₂ is H, and R₃ is

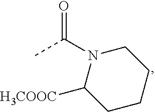

as shown by the following Formula (XII):

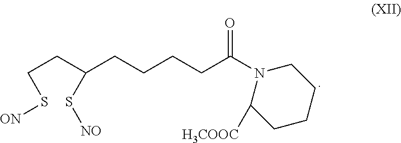

In another preferred embodiment of the invention, a compound of Formula (I) is provided where m equals 1, n equals 1, R₁ is H, R₂ is H, and R₃ is

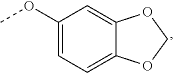

as shown by the following Formula (XIII):

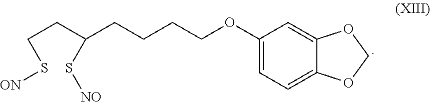

In another embodiment of the invention, a compound of Formula (I) is provided where m equals 1, n equals 1, R₁ is H, R₂ is H, and R₃ is

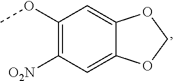

as shown by the following Formula (XIV):

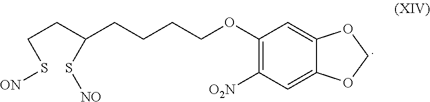

In other embodiments of the present invention, compounds useful in the invention with have the general Formula (XV) as follows:

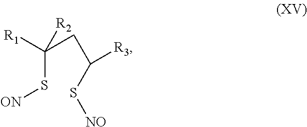

wherein:

R₁ and R₂ are independently selected from the group consisting of H, CH₃, and tert-butyl; and

R₃ is selected from the group consisting of CH₂CHCHCH₂COOCH₃, CH₂CHCHCHCHCOOH, and CHCHCHCHCOOCH₂CH₃.

In one preferred embodiment of the invention, a compound of Formula (XV) is provided where R₁ is methyl, R₂ is methyl, and R₃ is CH₂CHCHCH₂COOCH₃, as shown by the following Formula (XVI):

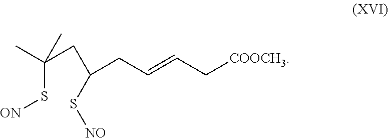

In another preferred embodiment of the invention, a compound of Formula (XV) is provided where R₁ is methyl, R₂ is methyl, and R₃ is CH₂CHCHCHCHCOOH, as shown by the following Formula (XVII):

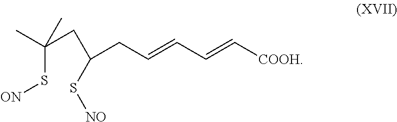

In another preferred embodiment of the invention, a compound of Formula (XV) is provided where R₁ is methyl, R₂ is methyl, and R₃ is CHCHCHCHCOOCH₂CH₃, as shown by the following Formula (XVIII):

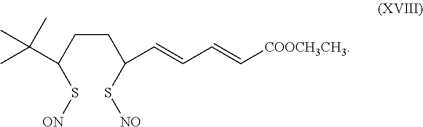

The foregoing compounds of the present invention are capable of serving as stable and effective NO-donating compounds. Typically, many S-nitroso compounds are not stable at room temperature or in the presence of light, and thus must be stored at temperatures of −20° C. or lower or in a dark environment to retain the NO group on the sulfur atom and, consequently, their biological activity. It has been determined, however, that the compounds of the present invention are generally stable at room temperature and are able to serve as efficient NO donor molecules after being stored at room temperature for an extended period of time. In this regard, it has been observed that, due to steric hindrance, a tertiary carbon adjacent to the sulfur atom promotes stabilization of the molecules and thus allows the compounds of the present invention to serve as stable NO-donating molecules. Additionally, after releasing NO, it is believed that the compounds regenerate lipoic acid and thus also serve as an effective source of lipoic acid.

In addition, and as indicated above, the compounds included herein are described with reference to formulas where one or more additional moieties can be incorporated into the core structure. In these embodiments, reference to the compounds of the present invention can include stereoisomers of the one or more moieties of the compounds. Such stereoisomers are representative of some embodiments of the compounds; however, the formulas and reference to the formulas disclosed herein are intended to encompass all active stereoisomers of the depicted compounds. Furthermore, the compounds of the present invention can, in some embodiments, contain one or more additional asymmetric carbon atoms, and can exist in raecemic and optically active forms. All of these other forms are contemplated to be within the scope of the present invention. As such, the compounds of the present invention can exist in stereoisomeric forms and the products obtained can thus be mixtures of the isomers.

In accordance with the present invention, all of the compounds described herein can be provided in the form of a pharmaceutically-acceptable salt or solvate, as would be recognized by one skilled in the art. A salt can be formed using a suitable acid and/or a suitable base. Suitable acids that are capable of forming salts with the compounds of the present invention include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, or the like. Suitable bases capable of forming salts with the compounds of the present invention include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine, and the like), and optionally substituted ethanolamines (e.g., ethanolamine, diethanolamine, and the like).

As used herein, the term “solvate” refers to a complex or aggregate formed by one or more molecules of a solute, e.g., a compound of the present invention or a pharmaceutically-acceptable salt thereof, and one or more molecules of a solvent. Such solvates are typically crystalline solids having a substantially fixed molar ratio of solute and solvent. Representative solvents include, but are not limited to, water, methanol, ethanol, isopropanol, acetic acid, and the like. When the solvent is water, the solvate formed is a hydrate. As such, the term “pharmaceutically-acceptable salt or solvate thereof” is intended to include all permutations of salts and solvates, such as a solvate of a pharmaceutically-acceptable salt of the present compounds.

In yet a further embodiment of the compounds of the present invention, and as described further below, pharmaceutical compositions are provided which comprise the compounds described herein and a pharmaceutically acceptable vehicle, carrier or excipient. For example, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone (POVIDONE™), hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

In one preferred embodiment, a sustained-release formulation of a compound of the present invention is provided that utilizes a polyanhydride-based technology. As will be recognized by those skilled in the art, polyanhydrides are a distinctive class of polymers for drug delivery because of their biodegradability and biocompatibility properties. In some embodiments, the release rate of polyanhydride-based formulations can be tuned over several folds by incorporating changes in the polymer structure. As such, in some embodiments of the sustained-release formulations of the presently-described compounds, the polymers employed to provide a sustained-release formulation are selected from poly[1,3-bis(p-carboxyphenoxy) propane, poly[1,3-bis(p-carboxyphenoxy)hexane-co-sebacic anhydride], poly[1,3-bis(p-carboxyphenoxy) methan-co-sebacic anhydride], and poly(fumaric anhydride). Apart from polyanhydride based formulations, in some embodiments, chitosan-based control release technology can be employed to provide a sustained-release formulation, as described further below.

Furthermore, liquid formulations of the compounds for oral administration can be prepared in water or other aqueous vehicles, and can contain various suspending agents such as methylcellulose, alginates, tragacanth, pectin, kelgin, carrageenan, acacia, polyvinylpyrrolidone, and include solutions, emulsions, syrups, and elixirs containing, together with the active components of the composition, wetting agents, sweeteners, and coloring and flavoring agents.

Various liquid and powder formulations can also be prepared by conventional methods for inhalation into the lungs of the subject to be treated. For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired compound and a suitable powder base such as lactose or starch.

Injectable formulations of the compounds can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compounds can be administered by the drip method, whereby a formulation including a pharmaceutical composition of the present invention and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically-acceptable oil base, such as an ester of a long chain fatty acid, (e.g., ethyl oleate).

In addition to the formulations described above, the compounds of the present invention can also be formulated as rectal compositions, such as suppositories or retention enemas, e.g., containing conventional suppository bases such as cocoa butter or other glycerides. Further, the compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt.

In some embodiments of the present invention, the compounds of the present invention may be incorporated into a nanoparticle. A nanoparticle within the scope of the invention is meant to include particles at the single molecule level as well as those aggregates of particles that exhibit microscopic properties. Methods of using and making a nanoparticle that incorporates a compound of interest are known to those of ordinary skill in the art and can be found following references: U.S. Pat. Nos. 6,395,253, 6,387,329, 6,383,500, 6,361,944, 6,350,515, 6,333,051, 6,323,989, 6,316,029, 6,312,731, 6,306,610, 6,288,040, 6,272,262, 6,268,222, 6,265,546, 6,262,129, 6,262,032, 6,248,724, 6,217,912, 6,217,901, 6,217,864, 6,214,560, 6,187,559, 6,180,415, 6,159,445, 6,149,868, 6,121,005, 6,086,881, 6,007,845, 6,002,817, 5,985,353, 5,981,467, 5,962,566, 5,925,564, 5,904,936, 5,856,435, 5,792,751, 5,789,375, 5,770,580, 5,756,264, 5,705,585, 5,702,727, and 5,686,113, each of which is incorporated herein by this reference.

Nanoparticles are frequently regarded as solid colloidal particles ranging in size from 10 nm to 1 μm, and can be built from macromolecular assemblies, in which an active compound or agent (e.g., a compound of the present invention) is dissolved, entrapped, encapsulated, or adsorbed or attached to the external interface to provide kinetic stability and rigid morphology. In some embodiments of the present invention, a bio-polymer-based nanoparticle formulation is utilized for efficient delivery of a compound of the presently-disclosed subject matter. In some embodiments, a formulation can be provided that utilizes chitosan/polyguluronate nanoparticles, poly(D,L-lactic acid)/ethyl acetate-based nanoparticles, PLGA-, PLGA:poloxamer-, or PLGA:poloxamine/dichloromethane-mediated nanoparticles, PEGylated polymeric micelles, or nanoparticles of albumin. As will be recognized by those of skill in the art, the preparation of nanoparticles as a composition vehicle will depend on the types of biopolymers employed in the process.

In one preferred embodiment of the present invention, a nanoparticle formulation can be provided that is derived from a chitosan/polyguluronate combination. Chitosan is a naturally existing polysaccharide composed of glucosamine and N-acetylglucosamine residues and can be derived by partial deacetylation of chitin, which is generally obtained from crustacean shells. Chitosan is known to be a biocompatible, low toxic, low immunogenic, and degradable by enzymes. In this regard, a nanoparticle formulation of the compounds of the present invention can be prepared by first dissolving chitosan glutamate in a suitable buffer, and, similarly, dissolving polyguluronate in a sodium sulfate buffer. The solutions can then be filtered through a micro-filter, and the nanoparticle formulations can then be prepared by adding the chitosan solution to an equal volume of the polyguluronate solution and then incubating the particles room temperature. In this regard, to incorporate a compound of the present invention into the nanoparticles, a desired amount of the compound, in a polar solvent, can be first added to the polyguluronate solution, and then the mixture can be combined with the chitosan solution. The resulting nanoparticles can then be incubated at room temperature before use or further analysis (see, e.g., Hoffman A S, The origins and evolution of “controlled” drug delivery systems, Journal of Controlled Release, 132 (2008), 153-163).

With further regard to the compounds of the present invention, it is again noted that the compounds of the present invention include nitric oxide derivatives of lipoic acid and, more particularly, nitric oxide derivatives of DHLA. As used herein, the term “derivative” refers to a chemically or biologically modified version of a chemical compound that is structurally similar to the parent compound and derivable from that parent compound. A “derivative” differs from an “analogue” in that a parent compound can be the starting material to generate a “derivative,” whereas the parent compound may not necessarily be used as the starting material to generate an “analogue.” Additionally, a derivative may or may not have different chemical or physical properties of the parent compound. For example, the derivative may be more hydrophilic or it may have altered reactivity as compared to the parent compound. In this regard, derivatization (i.e., modification) may involve substitution of one or more moieties within the molecule (e.g., a change in functional group). For example, a hydrogen may be substituted with a halogen, such as fluorine or chlorine, or, as another example, a hydroxyl group (—OH) may be replaced with a carboxylic acid moiety (—COOH).

As used herein, the term “derivative” also includes conjugates and prodrugs (i.e., chemically modified derivatives which can be converted into the original compound under physiological conditions) of a parent compound. For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). Further information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443, each of which is incorporated herein by this reference.

In some embodiments of the present invention, methods for making a compound of the present invention (i.e., a compound of Formula (I) or (XV)) are further provided. In one preferred embodiment, a method of making a compound of Formula (I) is provided that comprises: providing alpha lipoic acid or a derivative thereof; reducing the alpha lipoic acid or the derivative thereof to form dihydrolipoic acid or a dihydrolipoic acid derivative; exposing the dihydrolipoic acid or derivative thereof to nitric oxide for a time sufficient to create a nitroso-form of dihydrolipoic acid; and purifying the nitroso-form of dihydrolipoic acid.

In accordance with the present invention, methods for treating various diseases or disorders, or their underlying causes, using the presently-disclosed compounds are also provided, as described in further detail below. In one preferred embodiment, a method for treating a disease or disorder, in which the administration of a lipoic acid compound (e.g., dihydrolipoic acid) and an NO-donor compound is indicated, is provided that comprises administering to a subject an effective amount of a compound of the present invention, which includes a compound of Formula (I) or (XV), or pharmaceutically-acceptable salts or solvates thereof, to thereby treat the disease or disorder in the subject. In some embodiments, the disease or disorder is selected from angina, hypertension, diabetes, dyslipidemia, renal insufficiency, myocardial infarction, stroke, atherosclerosis, and the target organ damage that accompanies these various diseases and disorders.

As used herein, the terms “treatment” or “treating” relate to any treatment of a disease or disorder, including but not limited to prophylactic treatment and therapeutic treatment. As such, the terms “treatment” or “treating” include, but are not limited to: preventing a disease or disorder or the development of a disease or disorder; inhibiting the progression of a disease or disorder; arresting or preventing the further development of a disease or disorder; reducing the severity of a disease or disorder; ameliorating or relieving symptoms associated with a disease or disorder; and causing a regression of a disease or disorder or one or more of the symptoms associated with a disease or disorder.

In one preferred embodiment of the present invention, a method for treating hypertension is provided that comprises administering to a subject an effective amount of a compound of the present invention, which includes a compound of Formula (I) or (XV), or pharmaceutically-acceptable salts or solvates thereof, to thereby treat hypertension in the subject.

In another preferred embodiment of the present invention, a method for treating dyslipidemia is provided that comprises administering to a subject an effective amount of a compound of the present invention, which includes a compound of Formula (I) or (XV), or pharmaceutically-acceptable salts or solvates thereof, to thereby treat the dyslipidemia in the subject.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg=12 (Freireich, et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the present invention include, but are not limited to, systemic administration, parenteral administration (including intravascular, intramuscular, intraarterial administration), oral delivery, buccal delivery, rectal delivery, subcutaneous administration, intraperitoneal administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

Regardless of the route of administration, the compounds of the present invention are typically administered in amount effective to achieve the desired response. As such, the term “effective amount” is used herein to refer to an amount of the therapeutic composition (e.g., a composition comprising a compound of Formula (I) or (XV), and a pharmaceutically vehicle, carrier, or excipient) sufficient to produce a measurable biological response (e.g., a decrease in blood pressure or improved tissue blood flow). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

In certain embodiments of the methods of treating a disease or disorder in which the administration of a lipoic acid compound (e.g., dihydrolipoic acid) and an NO-donor compound is indicated, the compounds can be administered at a dose between about 10 mg/day and about 600 mg/day. In other embodiments, the compounds can be administered at about 100 mg/day and about 400 mg/day. In yet further embodiments, the compounds can be administered at a starting dose of 300 mg daily and can then be escalated in the absence of dose-limiting toxicity to a minimally effective amount.

For additional guidance regarding formulation and dose, see U.S. Pat. Nos. 5,326,902 and 5,234,933; PCT International Publication No. WO 93/25521; Berkow, et al., (1997) The Merck Manual of Medical Information, Home ed. Merck Research Laboratories, Whitehouse Station, N.J.; Goodman, et al., (2006) Goodman & Gilman's the Pharmacological Basis of Therapeutics, 11th ed. McGraw-Hill Health Professions Division, New York; Ebadi. (1998) CRC Desk Reference of Clinical Pharmacology. CRC Press, Boca Raton, Fla.; Katzung, (2007) Basic & Clinical Pharmacology, 10th ed. Lange Medical Books/McGraw-Hill Medical Pub. Division, New York; Remington, et al., (1990) Remington's Pharmaceutical Sciences, 18th ed. Mack Pub. Co., Easton, Pa.; Speight, et al., (1997) Avery's Drug Treatment: A Guide to the Properties, Choice, Therapeutic Use and Economic Value of Drugs in Disease Management, 4th ed. Adis International, Auckland/Philadelphia; and Duch, et al., (1998) Toxicol. Lett. 100-101:255-263, each of which are incorporated herein by reference.

In yet another embodiment of the therapeutic methods described herein, administering an effective amount of a compound of the present invention to a subject reduces an amount of oxidation of a low-density lipoprotein (LDL) in the subject. The effective amount of a therapeutic composition administered to a subject in accordance with the present invention to reduce LDL oxidation will vary depending on the subject's circumstances and the desired result to be achieved, but can readily be determined using routine experimentation.

Current research indicates that an abundance of reactive oxygen species in the vasculature of a subject results in an increased oxidation of proteins such as oxidized LDL (ox-LDL), which then initiates an inflammatory process and causes intimal damage to the arterial wall (See, e.g., Witztum J L, Steinberg D. J Clin Invest. 1991; 88:1785-1792). While the mechanisms of this damage are not yet established and may involve the inactivation of nitric oxide (NO) by oxygen-derived free radicals such as superoxide, the inflammatory response seen in these subjects has been observed to affect the gene expression of various inflammatory molecules, such as VCAM and tumor necrosis factor-alpha (TNF-α), which in turn can regulate the inflammatory process and promote foam cell formation (See, e.g., Rajagopalan S, Harrison D G. Circulation 1996; 94:240-243; Henninger D D, et al. Circ Res 1997; 81:274-281; Stannard A K, et al. Atherosclerosis 2001; 154:31-38; and, Libby P, et al. Curr Opin Lipidol 1996; 7:330-335). The reduction in NO levels along with an increase in ox-LDL may function as immunomodulators of the atherosclerotic process (See, e.g., Vergnani L, et al. Circulation 2000; 101:1261-1266.). Disclosed herein, however, are data showing that a compound of the present invention can effectively be used to significantly reduce the amount of LDL oxidation.

Various methods of measuring an amount of LDL oxidation are known to those of ordinary skill in the art and can be used in accordance with the present invention. For example, an amount of LDL oxidation can be measured by obtaining plasma samples from subjects, isolating the LDLs by ultracentrifugation, and then oxidizing the LDL to ox-LDL using a standard assay involving CuSO₄ (See, e.g., Zieden B, et al. Br J Clin Pharmacol. 1995; 39: 201-203). The lag time of oxidation, which indicates the susceptibility of LDL to oxidize, can then be measured using a spectrophotometer to allow the amounts of LDL oxidation occurring in a subject to be ascertained.

In another embodiment of the present invention, a method of improving vasodilation is provided whereby a subject in need of treatment is administered an amount of a compound in accordance with the invention that is effective to improve vasodilation in the subject. Again, the effective amount of a therapeutic composition administered to a subject in accordance with the present invention to improve vasodilation will vary depending on the subject's circumstances and the desired result to be achieved, but can readily be determined using routine experimentation.

Various methods of measuring the extent of vasodilation in a subject can be used in accordance with the present invention, including a non-invasive flow-mediated dilation technique, which uses high-resolution ultrasound to evaluate endothelial-dependent and endothelial-independent vasodilatation in the brachial artery. Briefly, that test stimulates the endothelium of the brachial artery in the arm to release nitric oxide, which then causes vasodilatation of the artery. The resulting vasodilatation can then be measured and quantified as a marker of endothelial function.

In other embodiments of the therapeutic methods disclosed herein, administering a composition of the present invention to the subject reduces an amount of inflammation in a subject, such as by reducing serum levels of an inflammatory molecule in a subject. As noted herein, recent evidence has indicated that an abundance of reactive oxygen species in the vasculature of a subject results in an increased oxidation of proteins such as oxidized LDL (ox-LDL), which then initiates an inflammatory process, causes intimal damage to the arterial wall, and effects the gene expression of a variety of inflammatory molecules. It is believed though that by administering a compound of the present invention to a subject, the serum levels of inflammatory molecules in the subject can be advantageously reduced to thereby reduce an amount of inflammation in a subject.

Various methods known to those skilled in the art can be used to determine a reduction of serum levels of inflammatory molecules in a subject. For example, in certain embodiments, the amounts of expression of an inflammatory molecule in a subject can be determined by probing for mRNA of the gene encoding the inflammatory molecule (e.g., PAI-1, VCAM-1, leptin, or adiponectin) in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, Calif.). Because the probes to which the nucleic acid molecules of the sample are bound are known, the molecules in the sample can be identified. In this regard, DNA probes for one or more of the mRNAs encoded by the inflammatory genes can be immobilized on a substrate and provided for use in practicing a method in accordance with the present invention.

With further regard to determining levels of inflammatory molecules in samples, mass spectrometry and/or immunoassay devices and methods can be used to measure the inflammatory molecules in samples, although other methods can also be used and are well known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Any suitable immunoassay can be utilized, for example, enzyme-linked immunoassays (ELISA), radioimmunoassays (RIAs), competitive binding assays, and the like. Specific immunological binding of the antibody to the inflammatory molecule can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

The use of immobilized antibodies or fragments thereof specific for the inflammatory molecules is also contemplated by the present invention. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

Mass spectrometry (MS) analysis can be used, either alone or in combination with other methods (e.g., immunoassays), to determine the presence and/or quantity of an inflammatory molecule in a subject. Exemplary MS analyses that can be used in accordance with the present invention include, but are not limited to: liquid chromatography-mass spectrometry (LC-MS); matrix-assisted laser desorption/ionization time-of-flight MS analysis (MALDI-TOF-MS), such as for example direct-spot MALDI-TOF or liquid chromatography MALDI-TOF mass spectrometry analysis; electrospray ionization MS (ESI-MS), such as for example liquid chromatography (LC) ESI-MS; and surface enhanced laser desorption/ionization time-of-flight mass spectrometry analysis (SELDI-TOF-MS). Each of these types of MS analysis can be accomplished using commercially-available spectrometers, such as, for example, triple quadropole mass spectrometers. Methods for utilizing MS analysis to detect the presence and quantity of peptides, such as inflammatory molecules, in biological samples are known in the art. See, e.g., U.S. Pat. Nos. 6,925,389; 6,989,100; and 6,890,763 for further guidance, each of which are incorporated herein by this reference.

With regard to the various therapeutic methods described herein, although certain embodiments of the methods disclosed herein only call for a qualitative assessment (e.g., the presence or absence of the expression of an inflammatory gene in a subject), other embodiments of the methods call for a quantitative assessment (e.g., an amount of reduction of LDL-oxidation in a subject or an amount of vasodilation in a subject). Such quantitative assessments can be made, for example, using one of the above mentioned methods, as will be understood by those skilled in the art.

The skilled artisan will also understand that measuring a reduction in the amount of a certain feature (e.g., LDL-oxidation) or an improvement in a certain feature (e.g., vasodilation) in a subject is a statistical analysis. For example, a reduction in an amount of LDL-oxidation in a subject can be compared to control level of LDL-oxidation, and an amount of LDL-oxidation of less than or equal to the control level can be indicative of a reduction in the amount of LDL-oxidation, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

The compounds of the present invention are designed to include the beneficial properties of lipoic acid and, in particular, DHLA with those of the nitric oxide. As such, it is believed that the presently-disclosed compounds will be useful as potent antioxidants, anti-inflammatory compounds, and as mitochondrial protective agents. Consequently, it is thus further contemplated that the presently-disclosed compounds can be useful for the treatment of a number of diseases and disorders where the beneficial properties of lipoic acid and nitric oxide are indicated.

For example, it is contemplated that the present compounds will be particularly useful in the treatment of diabetes. In this regard, it is contemplated that the compositions of the present invention will be useful for reducing oxidative stress, improving insulin signaling, treating diabetic complications that occur from overproduction of reactive oxygen and nitrogen species, and preventing the age-dependent development of hyperglycemia, hyperinsulinemia, dyslippidemia, and plasma markers of oxidative stress. Furthermore, it is also contemplated that the present compositions will be useful for preventing the mitochondrial decay that has been postulated to account for a considerable portion of the metabolic dysfunction that occurs in diabetes.

As another example, it is also contemplated that the present compositions will be useful for treating hypertension, myocardial infarction, stroke, and atherosclerosis, as well as the target organ damage that accompanies these various diseases and disorders. In this regard, it is contemplated that the present compounds will be capable of improving endothelial dysfunction by, for example, improving endothelium-dependent vasorelaxation, reducing adhesion molecules and chemokines, lowering serum triglycerides, and lowering inflammatory gene expression. In addition, it is contemplated that the present compositions will be capable of improving renal insufficiency and/or slowing the deterioration of kidney function in diabetes and hypertension by, for example, reducing or preventing the progression of microalbuminuria to subsequent overt proteinuria and renal failure.

In yet a further application of the present invention, it is contemplated that the compounds described herein will be useful in treating angina by making NO molecules available to the endothelium for vasodilation, thereby reversing or inhibiting coronary vasospasms that may occur in a subject.

In still another application of the present invention, it is contemplated that, because the compounds of the present invention include S-nitrosothiols, these compounds will be particularly beneficial in treating various diseases. For example, the S-nitrosothiol compounds of the present invention are expected to have an advantage over other classes of NO-donor molecules due to tissue selectivity, as S-nitrosothiols are commonly selective for arteries over veins and are potent anti-platelet agents, inhibiting aggregation at doses that do not influence vascular tone. As another example, it is expected that the ability of S-nitrosothiols to directly transfer NO species will allow biological activity to be passed on through a chain of other thiols without the release of free NO. This mechanism of bioactivation can thus make the S-nitrosothiol compounds of the present invention less susceptible to conditions of oxidative stress by effectively protecting the NO moiety from attacking oxygen radicals. As still another example, the S-nitrosothiol compounds of the present invention are expected to be efficient vasodilators and can potentially be used to treat endothelial dysfunction in a subject in need of such treatment. Furthermore, when the S-nitrosothiol compounds are used along with bio-compatible polymers, it is expected that this will reduce a number of unwanted complications such thrombosis and restenosis without the need for systemic administration of heparin or potent anti-platelet agents. Additionally, and as yet another example, S-nitrosothiol compounds have been shown to have neuroprotective properties via regulation of antioxidant and apoptotic enzymes, consequently, it is expected that the S-nitrosothiol compounds of the present invention can be used in delaying the progression of neurodegenerative disorders or even encouraging neuro-regeneration. Finally, it is also contemplated that administering a S-nitrosothiol compound of the present invention will promote wound healing properties, as supplementation of endogenous S-nitrosothiols has been suggested to encourage wound healing in subjects.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The embodiments of the presently-disclosed subject matter as set forth herein are subject to modifications, and other modified embodiments within the scope of the invention will be evident to those of ordinary skill in the art after a study of the information provided in this document. The information provided in this document, and particularly the specific details of the described exemplary embodiments, is provided primarily for clearness of understanding and no unnecessary limitations are to be understood therefrom.

Further, while the terms used in the application are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the present invention belongs. Although any methods, devices, and materials similar or equivalent to those described herein can be used in the practice or testing of the presently-disclosed subject matter, representative methods and materials have been described herein above.

Additionally, following long-standing patent law convention, the terms “a”, “an”, and “the” refer to “one or more” when used in this application, including the claims. Thus, for example, reference to “an inflammatory molecule” includes a plurality of such molecules, and so forth. Also, unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the present invention.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis and Characterization of a Dinitroso-Derivative of Dihydrolipoic Acid

To synthesize a dinitroso-derivative of dihydrolipoic acid (DHLA), two sources were used to initially obtain DHLA: 1) a commercial DHLA and 2) a DHLA that was created via a reduction of lipoic acid with borohydride or with other thiols. In the latter case, DHLA was extracted with solvents before use and dried. The chemicals and reagents described in the synthesis procedures below, including: lipoic acid, DHLA, sodium borohydride, sodium nitrite, cysteine, β-mercaptoethanol, bovine serum albumin (BSA) and other common reagents, were purchased from Sigma Chemical Company (St. Louis, Mo.).

In the synthesis procedure, nitric oxide (NO) was generated by the reaction of sodium nitrite with dilute hydrochloric acid (HCl). In a typical reaction, excess sodium nitrite (about 200 mg) was treated with 1 ml of 6N hydrochloric acid, with the volume and normality of the HCl being adjusted based on the use and size of the separatory funnel that was employed. DHLA (10 mg) in ethyl alcohol (100 μl) was cooled in a dry ice-acetone bath. The sample was frequently mixed by vortexing, and the NO released in the sodium nitrite/HCl reaction was bubbled through the DHLA solution. The formation of a pink solution was immediately seen. When darkening of the color ceased to occur, the bubbling was stopped.

In these procedures, a solution that included no DHLA was observed to be yellow in color. In contrast, a solution that contained only DHLA was observed to be colorless, whereas the solution that contained both the nitrite/HCL and the DHLA was observed to be pink in color. The later product was further observed to be distinct from DHLA or lipoic acid and migrated as a single product on thin layer chromatography. The product also gave a characteristic visible spectrum with two peaks at 350 and 545 nm, and the reaction described above appeared to be 100% complete with no starting material remaining.

Specifically, to characterize the resulting nitric oxide derivative of DHLA, high-performance liquid chromatography was first performed and it was observed that no lipoic acid remained in the reaction. Also, because the nitroso-derivatives had a characteristic visible spectrum due to the pink color, visible spectral analysis of the product was performed and a characteristic spectrum was observed with a peak around 545 nm (FIG. 3). Finally, sulfhydral content was also measured before and after each reaction, and practically no free sulfhydral groups could be seen after the reaction.

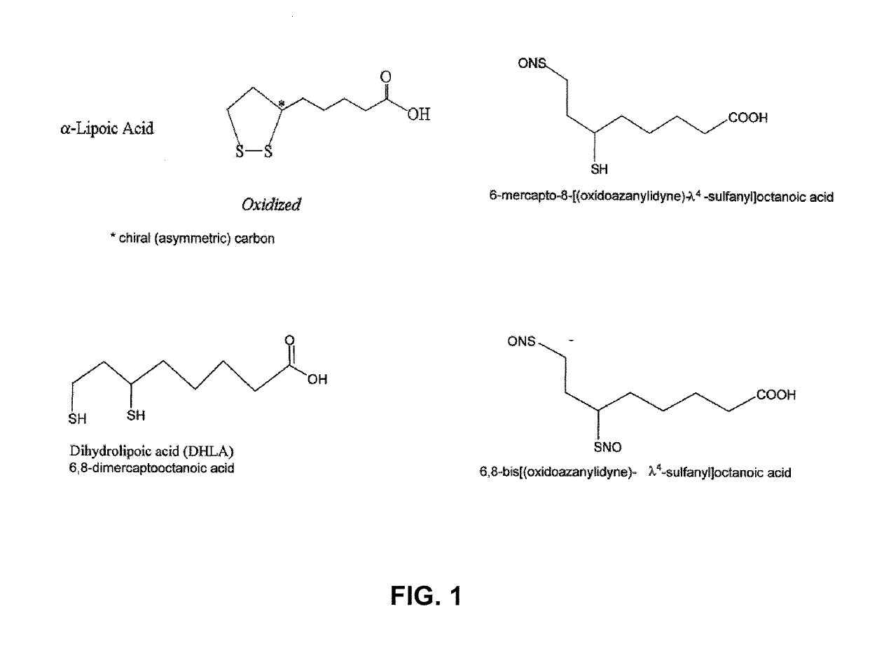

Two types of products were feasible from these sets of reactions, including: a dinitrosylated product and a mononitrosylated product, as shown in FIG. 1. It was believed that the formation of a thiolactone under acidic conditions would favor a mononitroslylated product. As such, in order to make a specific dinitrosylated product, lipoic acid was converted into its methyl ester and then the product was reduced to generate the thiols prior to reacting it with nitric oxide. That product had an intense purple color and no free thiols remained in the product as determined by a subsequent 5,5′-dithio-bis(2-nitrobenzoic acid (DTNB) reaction.

In summary, from these synthesis procedures, it was observed that the reaction of NO with reduced lipoic acid (dihydrolipoic acid; DHLA) generated a pink solution that showed a typical spectrum of nitrosothiols absorbing at 545 nm and 350 nm. A reddish solid was produced and that color was not seen with lipoic acid as that compound does not have free thiol groups. Furthermore, it was also observed that other reduced thiols (cysteine, glutathione), which were used as controls, generated similar S-nitroso thiol compounds. NO generated from the decomposition of other NO donors also generated nitroso-DHLA when incubated with DHLA. Finally, it was observed that the DHLA-NO solution stored at room temperature decomposed, losing color within hours. However, DHLA-NO stored at −80° C. retained its color up for up to 3 months without any loss of absorbance at OD 545 nm. DHLA-NO was also observed to be stable at −20° C. for at least one month, without any loss of absorbance at OD 545 nm.

### Example 2

### Nitration of Bovine Serum Albumin by a Dinitroso-Derivative of Dihydrolipoic Acid

To determine the ability of the dinitroso-derivative of DHLA (6,8-bis[(oxidoazanylidyne)-λ⁴-sulfanyl]octanoic acid or “NO-DHLA”) to donate nitric oxide (NO), bovine serum albumin (BSA) was first prepared in water at 25 mg/ml. In 100 μl of alcohol, increasing concentrations of BSA (15 to 30 μl) were then mixed with increasing concentrations of the NO-DHLA (0 to 15 μl) and incubated for 1 hr. Table 1 represents the incubation:

TABLE 1

| NO-DHLA (μl) | BSA (μl) |
|---|---|
| 0 | 30 |
| 15 | 15 |
| 10 | 20 |
| 7.5 | 22.5 |
| 6 | 24 |

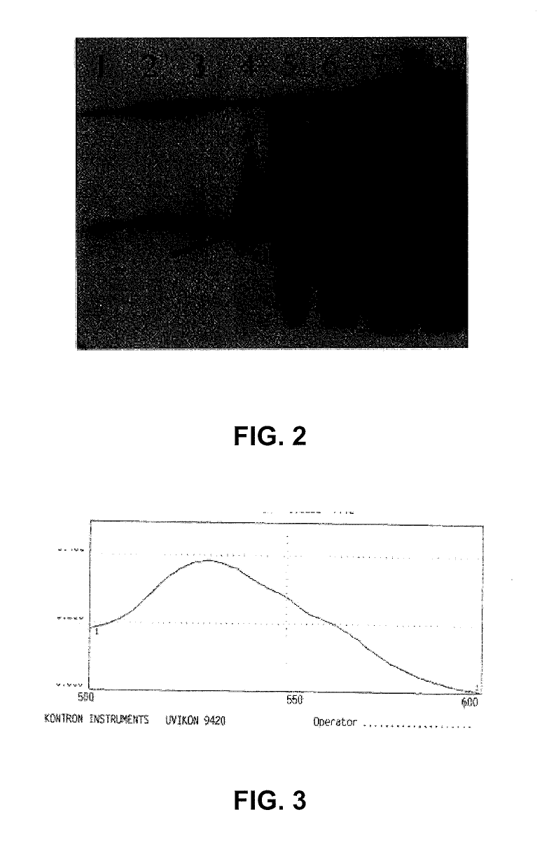

At the end of the incubation, samples were the separated by SDS-PAGE electrophoresis with along with markers of molecular weight. At the end of the electrophoresis, the blot was subsequently transferred on to a nitrocellulose membrane and western blotting was performed using anti-nitrotyrosine antibodies at a 1:1000 dilution (Oxford Biomedical Research, Rochester Hills, Mich.). Non-specific sites on the nitrocellulose were blocked with milk protein for 16 h at 4° C. and then overlaid with a polyclonal anti-nitrotyrosine rabbit antibody. Peroxidase conjugated anti-rabbit IgG was used as secondary antibody. A representative western blot is shown in FIG. 2, where: lane 1 includes a molecular weight marker; lanes 2-4 include control samples with only the BSA at 0, 15, and 30 μl, respectively, without any NO-DHLA; lane 5 includes 15 μl of both the BSA and the NO-DHLA; lane 6 includes 10 μl of NO-DHLA and 20 μl of the BSA; lane 7 includes 7.5 μl of NO-DHLA and 22.5 μl of the BSA; and, lane 8 includes 6 μl of NO-DHLA and 24 μl of the BSA.

Tyrosine amino acids of proteins are vulnerable to nitration and form nitro-tyrosines, which can been taken as in vivo evidence for nitric oxide involvement. (See, e.g., MacMillan-Crow L A, et al., Nitration and inactivation of manganese superoxide dismutase in chronic rejection of human renal allografts. Proc Natl Acad Sci USA. 1996 Oct. 15; 93(21):11853-8.)) Upon analysis of the results of the foregoing experiments, it was thus observed that untreated albumin had no detectable nitrotyrosine residues, whereas the samples that were incubated with NO-DHLA acid showed an intense reactivity with the antibody indicating the NO-DHLA effectively functioned as an NO-donor compound.

### Example 3

### Inhibition of Low-Density Lipoprotein Oxidation by a Dinitroso-Derivative of Dihydrolipoic Acid

To determine whether the dinitroso-derivative of dihydrolipoic acid (6,8-bis[(oxidoazanylidyne)-λ⁴-sulfanyl]octanoic acid or “NO-DHLA”) was capable of inhibiting low-density lipoprotein (LDL) oxidation, LDLs were incubated in ethyl alcohol alone or with NO-DHLA. A spectral analysis of these solutions was then performed at an O.D. of 234, as absorbance at that portion of the spectrum is indicative of the formation of the oxidized fatty acid component of LDL.

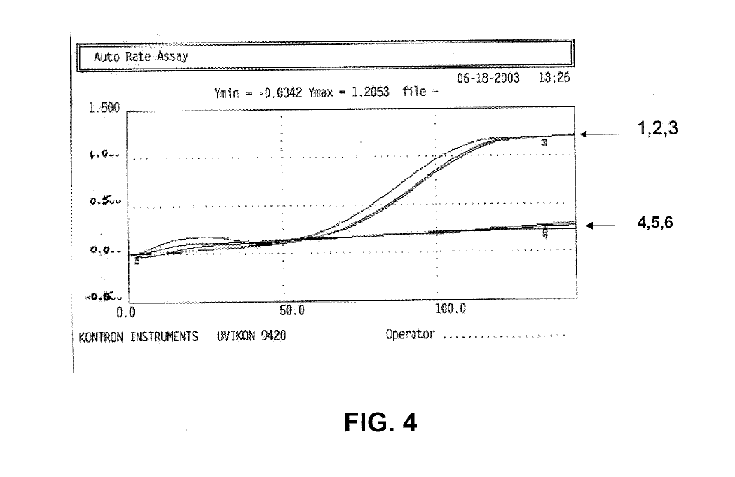

FIG. 4 shows a representative spectra of the results from these experiments where samples 1, 2, and 3 represented control sample including LDLs in 1, 2.5, and 5 μl of ethyl alcohol, respectively, and where samples 4, 5, 6 represented LDLs that had been combined with 10, 25, and 50 μM of the NO-DHLA in 1, 2.5, and 5 μl of ethyl alcohol, respectively. As shown in the graph of FIG. 4, where the X-axis is the time in minutes and the Y-axis is the O.D. at 234, the NO-DHLA effectively prevented or significantly reduced the oxidation of LDL at the concentrations measured. As such, it is believed that nitric oxide derivatives of DHLA can have a significant effect on atherosclerosis, diabetes, hypertension, and inflammation, all of which have been shown to be key elements of cardiovascular disease.

### Example 4

### Synthesis of 5-(5,7-bis(nitrososulfanyl)heptyloxy)benzo[d][1,3]dioxole

To synthesize 5-(5,7-bis(nitrososulfanyl)heptyloxy)benzo[d][1,3]dioxole, or the dinitroso-derivative of 3,4-methylenedioxyphenyl lipoate, a three step reaction was utilized. In the first step of the reaction, O-Lipoyl methylenedioxyphenol (3,4-methylenedioxyphenyl lipoate) was synthesized from methylenedioxyphenol and dl-lipoic acid. Briefly, 3,4-methylenedioxyphenyl lipoate was first made by treating lipoic acid and methylenedioxyphenol with a halogenated solvent under nitrogen atmosphere. In a 250 mL round bottom flask, 4.5 g (0.033 mole) of 3,4-methylenedioxyphenol, 5 g (0.4 mole) of dimethylamino-pyridine, and 120 mL of dichloromethane were combined and stirred well for 15 minutes. 7.4 g (0.036 mole) of alpha lipoic acid was then added in portions over a period of 10 minutes. N-(3-dimethylaminopropyl)-N′-ethylcarbodiimide hydrochloride (EDCI), 8.6 g (0.8 mole), was then added to the solution over a period of 45 minutes, and stirring was continued over night. After approximately 24 hours of reaction time, thin layer chromatography (3:1 hexane:ethyl acetate solvent system) of the crude product was checked with the starting materials to determine the formation of the expected product and the disappearance of starting material, 3,4-methylenedioxyphenol. After the reaction was determined to be completed, the dichloromethane was then evaporated under reduced pressure, and a thick yellow mass was obtained, which was subsequently directly purified on flash column chromatography using a 3:1 hexane:ethyl acetate solvent system.

Slow elution was then performed to get the product to a higher purity. Briefly, the yellow fluffy solid that was obtained was re-purified by a crystallization method. The crystallization was performed using 90% ethanol and the procedure involved a slow addition of the compound (approximately 2 g) over a period of 10 minutes into a warm ethanol (approximately 35 mL) with continuous stirring. After complete dissolution of the compound, the pale yellow solution was then filtered quickly and set aside for slow crystallization over night. The resulting shining and fluffy solid was then filtered and dried using a vacuum dryer, and the total yield was 65-73%. A schematic representation of the foregoing portion of the reaction is as follows:

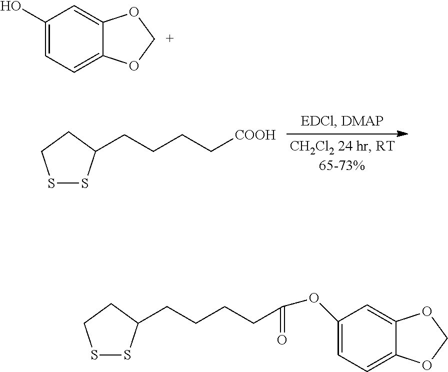

The crystallized compound was subsequently characterized by high resolution proton NMR to confirm the formation of the intermediate in the reaction procedure. The typical proton chemical shift values of the intermediate product were observed to be (CDCl₃): 6.77-6.75 (1H, doublet), 6.59-6.58 (1H, doublet), 6.52-6.49 (1H, doublet of doublet), 5.97 (2H, singlet), 3.61-3.57 (1H, multiplet), 3.19-3.10 (2H, multiplet), 2.56-2.52 (2H, triplet), 2.51-2.45 (2H, multiplet), 1.95-1.86 (2H, multiplet), 1.79-1.73 (4H, multiplet), 1.58-1.53 (2H, multiplet).

In the second step of the synthesis procedure, sodium borohydride was used to reduce 3,4-methylenedioxyphenyl lipoate to obtain 3,4-methylenedioxyphenyl dihydrolipoate. A schematic representation of this portion of the reaction procedure is as follows:

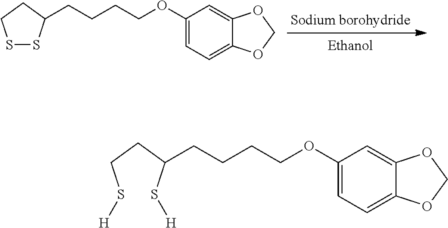

Briefly, 3,4-methylenedioxyphenyl lipoate (0.36 g) was dissolved in 25 mL of ethanol and stirred well for 5 minutes. Sodium borohydride, 0.149 g (1 mM) was then added to the solution by portions over a 2 hour period. Next, in order to derive the crude product, the ethanol was evaporated under reduced pressure, followed by treatment with saturated ammonium chloride (15 mL). The organic compound in the aqueous phase was extracted with dichloromethane (2×50 mL) and evaporated to dryness under reduced pressure.

In the third step of synthesis procedure, the dinitroso-derivative of 3,4-methylenedioxyphenyl dihydrolipoate was formed. A schematic representation of this portion of the reaction procedure is as follows:

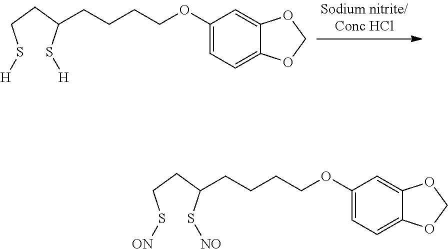

Briefly, the resultant dithiol intermediate prepared in the second step was dissolved in ethanol (25 mL) and cooled to −20° C. using dry ice and acetone. Simultaneously, nitric oxide gas was generated by reacting sodium nitrite and concentrated hydrochloric acid. The nitric oxide gas was then passed through the dithiol solution for two hours. The solution turned a deep pink color which is characteristic of an S-nitroso compound. UV-Visible spectrum analysis of this deep pink solution showed two characteristic absorption peaks at 330 and 540 nm. The product was stored at −80° C. and was determined to have the following chemical structure:

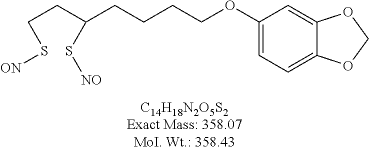

### Example 5

### Synthesis of 5-(5,7-bis(nitrososulfanyl)heptyloxy)-6-nitrobenzo[d][1,3]dioxole

To synthesize 5-(5,7-bis(nitrososulfanyl)heptyloxy)-6-nitrobenzo[d][1,3]dioxole, or the dinitroso derivative of 3,4-methylenedioxyphenyl-6-nitro lipoate, a three step synthesis procedure was utilized. In the first step of the synthesis procedure, nitromethylenedioxyphenol and alpha lipoic acid were reacted as schematically represented below:

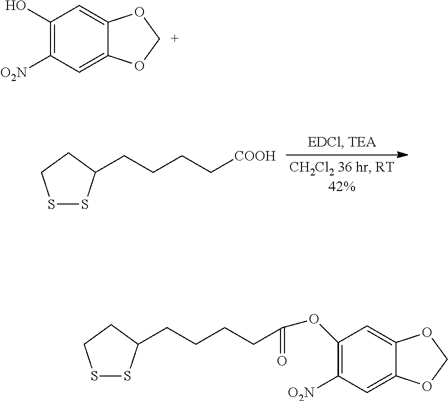

Briefly, in a 100 mL round bottom flask, 0.17 g (0.001 mole) of 3,4-methylenedioxy 6-nitrophenol and 0.1 g (140 μl; 0.2 mole) of triethylamine and 50 mL of dichloromethane were combined and stirred well for 10 minutes. The solution became reddish orange color. Alpha lipoic acid 0.25 g (0.0015 mole) was then added over a period of 5 minutes. N-(3-Dimethylaminopropyl)-N′-ethylcarbodiimide hydrochloride (EDCI), 0.4 g (0.002 mole) was subsequently added to the solution over a period of 15 minutes and the solution was allowed to continue to stir over night. After about 36 hours of reaction time, thin layer chromatography (20% hexane:ethyl acetate solvent system) of the crude product was checked with the starting materials to check the formation of the desired intermediate product and the disappearance of the starting material, 3,4-methylenedioxy 6-nitrophenol. After it was determined that the reaction had completed, the dichloromethane was evaporated under reduced pressure. A thick yellow mass was obtained, and was directly purified on preparative thin layer chromatography using a 20% hexane:ethyl acetate solvent system. The appropriate band was scratched out from the plate and was then carefully eluted with ethylacetate.

A pale yellow solid, which was obtained, was subsequently re-purified by a crystallization method. The crystallization was performed using 95% ethanol and the procedure involved a slow addition of the compound (approximately 50 mg) over a period of 5 minutes into warm ethanol (approximately 10 mL) with continuous stirring. After complete dissolution of the compound, the resultant solution was filtered quickly and set aside for slow crystallization over night. The resulting yellow solid was then filtered and dried using a vacuum dryer. The total yield was 42%. The crystallized intermediate product was subsequently characterized using high resolution proton NMR and the typical proton chemical shift values of the product were (CDCl₃): 7.59 (1H, singlet), 6.45 (1H, singlet), 6.15 (2H, singlet), 3.68-3.60 (1H, multiplet), 3.21-3.13 (2H, multiplet), 2.66-2.64 (2H, triplet), 2.52-2.47 (2H, multiplet), 1.97-1.92 (2H, multiplet), 1.84-1.75 (4H, multiplet), 1.74-1.58 (2H, multiplet.

In the second step of the synthesis procedure, sodium borohydride was used to reduce 3,4-methylenedioxyphenyl 6-nitro lipoate to obtain 3,4-methylenedioxyphenyl 6-nitro dihydrolipoate, as shown in the following schematic representation:

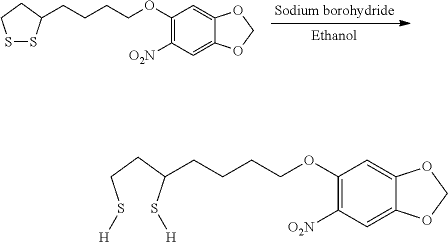

Briefly, 3,4-methylenedioxyphenyl 6-nitro lipoate (0.4 g) was dissolved in 25 mL of ethanol and stirred well for 5 minutes. Sodium borohydride, 0.149 g (1 mM) was then added to the solution by portions over a 2 hour period. Next, in order to derive the crude product, the ethanol was evaporated under reduced pressure, followed by treatment with saturated ammonium chloride (15 mL). The organic compound in the aqueous phase was extracted with dichloromethane (2×50 mL) and evaporated to dryness under reduced pressure. The final dihyro compound was obtained as pale yellow semi-solid which was used directly for the next step in the synthesis procedure, without additional purification.

In the third step of the synthesis procedure, the dinitroso derivative of 3,4-methylenedioxyphenyl 6-nitro dihydrolipoate was formed, as shown in the following schematic representation:

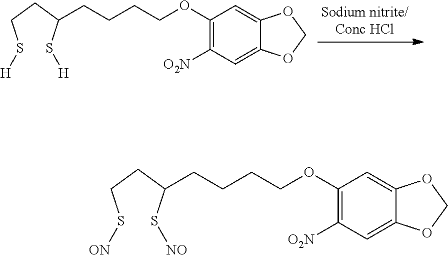

Briefly, the resultant dithiol intermediate prepared in the second step was dissolved in ethanol (25 mL) and cooled to −20° C. using dry ice and acetone. Simultaneously, nitric oxide gas was generated by reacting sodium nitrite and concentrated hydrochloric acid. The nitric oxide gas was then passed through the dithiol solution for two hours. The solution turned a deep pink color which is characteristic of an S-nitroso compound. UV-Visible spectrum analysis of this deep pink solution showed two characteristic absorption peaks at 330 and 540 nm. The product was stored at −80° C. and was determined to have the following chemical structure:

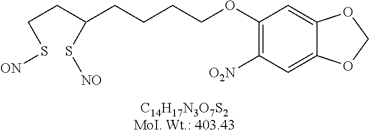

### Example 6

### Synthesis of 1-(6,8-bis(nitrososulfanyl)octanoyl)piperidine-2-carboxylic acid

The synthesis of 1-(6,8-bis(nitrososulfanyl)octanoyl)piperidine-2-carboxylic acid was performed by a three step procedure. The first step of the procedure is shown in the following schematic representation:

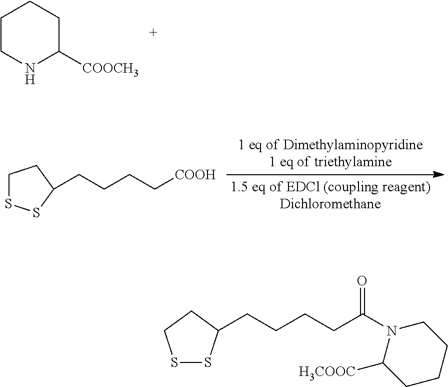

The first step of the procedure began with the synthesis of (DL)-pipecolinyl methyl ester lipoic acid from (DL)-pipecolinyl methyl ester and (DL)-alpha-lipoic acid. Briefly, the synthesis of (DL)-pipecolinyl methyl ester lipoic acid was completed by combining (DL)-pipecolinic acid methyl ester and (DL)-alpha-lipoic acid using a suitable coupling reagent, as shown in the scheme above. The first step of the reaction was carried out under a nitrogen atmosphere. (DL)-alpha-lipoic acid, 0.206 g (1 mM); (DL)-pipecolinic acid methyl ester hydrochloride, 0.166 g (1 mM); dimethylaminopyridine, 0.122 g (1 mM); and triethylamine, 0.101 g (1 mM, 0.140 mL) were dissolved in methylene chloride (35 mL) and were combined in a 100 mL round bottom flask. The contents of the flask were stirred well for 5 minutes at room temperature. The coupling reagent EDCI, 0.287 g (1.5 mM) was added in portions over a period of 1.5 hours while the contents of the flask were simultaneously stirred. Completion of the reaction was monitored using thin layer chromatography by comparing the disappearance of the starting materials and the formation of the new product. After stirring overnight, the methylene chloride was evaporated under reduced pressure using a rotary evaporator. The resultant crude product was purified using fluorescent preparative thin layer chromatography with a ratio of 90:5:10 ethylacetate:hexane:methanol. The desired compound band was then scraped and the compound was extracted from the band by continuously eluting with ethylacetate (350 mL). The solvent was evaporated to dryness under a vacuum. A pale yellow, highly viscous liquid was obtained in a 59% yield, and was determined to be (DL)-pipecolinyl methyl ester lipoic acid.

In the second step of the reaction procedure, (DL)-Pipecolinyl lipoic acid was synthesized by de-esterification of (DL)-pipecolinyl methyl ester lipoic acid in 1M ethanolic potassium hydroxide under reflux condition, as shown in the schematic representation below.

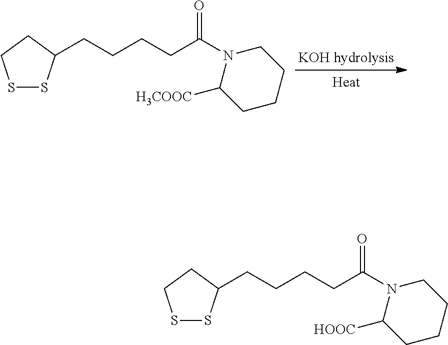

Briefly, the reaction was carried out under nitrogen atmosphere and (DL)-pipecolinyl methyl ester lipoic acid, 0.158 g (0.5 mM) was placed in a 50 mL round bottom flask equipped with reflux condenser. 25 mL of 1 mM ethanolic potassium hydroxide was added and the mixture was allowed to reflux for 24 hours. Progress of the reaction was monitored by thin layer chromatography. After the overnight reflux, ethanol was removed under reduced pressure followed by the addition of 30 mL of water and extracted the aqueous phase with dichloromethane (2×25 mL). Aqueous phase was carefully transferred to 100 mL conical flask, cooled well on crushed ice and acidified with 1 N hydrochloric acid till pH of the solution is acidic. The aqueous phase was then extracted with dichloromethane (2×50 mL), washed with brine and dried over anhydrous magnesium sulphate and filtered. Solvent was evaporated under reduced pressure to get yellow colored semi-solid 75% yield that was determined to be (DL)-pipecolinyl lipoic acid.

In the third step of the reaction procedure, 1-6, 8-dimercaptooctanyl) piperidine-2-carboxylic acid was first synthesized from pipecolinyl lipoic acid, as shown below:

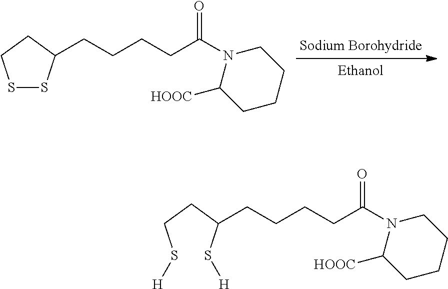

To perform this step of the reaction procedure, L-pipcolinyl lipoic acid, 0.165 g, was first dissolved in 25 mL of ethanol and stirred well for 5 minutes. Sodium borohydride (37 mg) was then added to stepwise to the solution over a 2 hour period. Next, in order to derive the crude product, the ethanol was evaporated under reduced pressure, followed by treatment with saturated ammonium chloride (15 mL). The organic compound in the aqueous phase was extracted with dichloromethane (2×50 mL) and evaporated to dryness under reduced pressure. The resultant dimercapto-derivative was purified on column chromatography to get the compound in a purified form.

The characteristic feature of thiol compounds includes the formation of a nitroso-derivative upon reaction with nitric oxide gas. As such, the resulting dithiol compound was tested to confirm its presence by reacting the compound with nitric oxide gas. Again, briefly, the intermediate was dissolved in ethanol (25 mL) and cooled to −20° C. using dry ice and acetone. Simultaneously, nitric oxide gas was generated by reacting sodium nitrite and concentrated hydrochloric acid. The nitric oxide gas was then passed through the dithiol solution for two hours to achieve the reaction shown below.

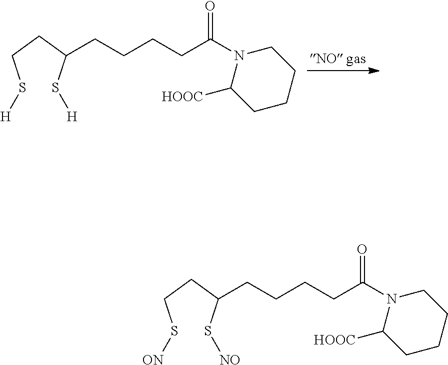

During this reaction, the solution turned a deep pink color which is characteristic of an S-nitroso compound. UV-Visible spectrum analysis of this deep pink solution showed two characteristic absorption peaks at 330 and 540 nm, confirming the presence of the dithiol compound. The compound was subsequently determined to have the following chemical structure.

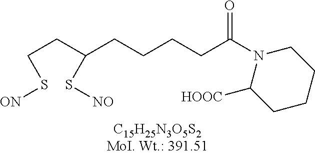

### Example 7

### Synthesis of (S)-1-(6,8-bis(nitrososulfanyl)octanoyl)pyrrolidine-2-carboxylic acid

To synthesize (S)-1-(6,8-bis(nitrososulfanyl)octanoyl)pyrrolidine-2-carboxylic acid, a three-step procedure was used starting from optically-active L-proline methyl ester and alpha lipoic acid as shown below.

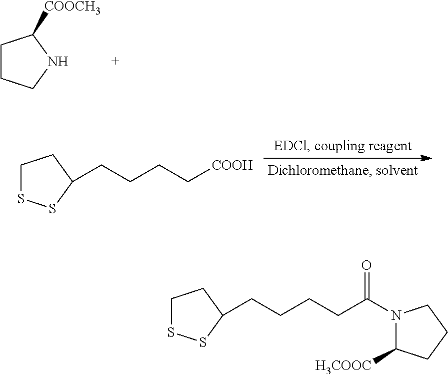

In the first step of reaction procedure, L-prolyl methyl ester lipoic acid was synthesized by coupling L-proline methyl ester and lipoic acid using a suitable coupling reagent. Briefly, the first step of the reaction was carried out under a nitrogen atmosphere. Lipoic acid, 0.206 g (1 mM), L-proline methyl ester hydrochloride, 0.166 g (1 mM), 1 equivalent of dimethylaminopyridine (DMAP) 0.122 g (1 mM), and triethylamine 0.101 g (0.140 mL) were combined in a 100 mL round bottom flask. The contents of the flask were dissolved in methylenechloride (40 mL) and stirred well for 10 minutes at room temperature. The coupling reagent, 1-ethyl-3-(3-dimethylaminopropyl) carbodiimide (EDCI), 0.287 g (1.5 mM) was added in portions over a period of 3 hours while the contents of the flask were simultaneously stirred. The completion of the reaction was monitored using thin layer chromatography by comparing the disappearance of starting materials and the formation of the new product.

After overnight stirring, the methylenechloride was evaporated under reduced pressure using a rotary evaporator. The resultant crude product was purified using fluorescent preparative thin layer (FPTL) chromatography with a ratio of 90:5:10 ethylacetate:hexane: methanol. The required compound band was scraped and the compound was extracted from it by continuously eluting with ethylacetate (350 mL). The solvent was evaporated to dryness and the last traces of solvent were removed under a vacuum pump. A yellow semi-solid was obtained in a 62% yield. The compound was characterized using proton nuclear magnetic resonance (NMR) spectroscopy, and peaks were assigned based on the compound's chemical shift values. The mass of the compound was determined to be 318.1 (M+1), and, at this point in the reaction, the compound was determined to be L-prolyl methyl ester lipoic acid.

In the second step of the reaction procedure, L-prolyl lipoic acid was synthesized by the de-esterification of L-prolyl methyl ester lipoic acid in 1M ethanolic potassium hydroxide under reflux conditions, as shown in the schematic below.

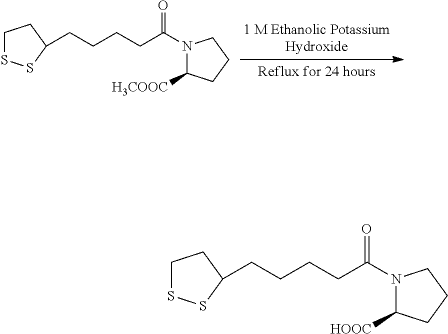

Similar to the first portion of the reaction procedure, this reaction step was also carried out under a nitrogen atmosphere. Briefly, L-prolyl methyl ester lipoic acid, 0.158 g (0.5 mM) was placed in a 50 mL round bottom flask that was equipped with a reflux condenser. 25 mL of 1 mM ethanolic potassium hydroxide was added and set to reflux for 24 hours. Progress of the reaction was monitored by thin layer chromatography by comparing the disappearance of starting materials and the formation of the new product.

After overnight reflux, ethanol was then removed from the reaction mixture under reduced pressure and the removal of the ethanol was followed by the addition of 30 mL of water. The aqueous phase was then extracted with dichloromethane (2×25 mL) and carefully transferred to a 100 mL conical flask, cooled well on crushed ice and acidified with 1 N hydrochloric acid until the pH of the solution became acidic. The aqueous phase was then extracted with dichloromethane (2×50 mL), washed with brine, dried over anhydrous magnesium sulfate, and filtered. The solvent was evaporated under reduced pressure, and a yellow colored semi-solid was obtained in a 75% yield. The resulting compound was characterized using proton NMR spectroscopy, and peaks were assigned based on the compound's chemical shift values. The mass of the compound was 326.1 (M+23), 607.3 (Dimer), where M+23 indicates a sodium adduct (Na MW=23), and, at this point in the reaction, the compound was determined to be L-prolyl lipoic acid.

In the third step of the reaction procedure, the disulfide portion of L-prolyl lipoic acid was converted into a dithiol moiety to produce 1-(6,8-dimercaptooctanyl) pyrrolidine-2-carboxylic acid, as shown in the following schematic.

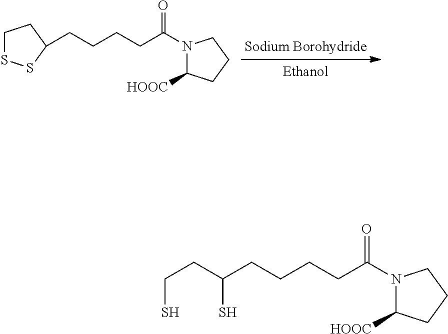

To perform this conversion of L-prolyl lipoic acid into a dithiol derivative, L-prolyl lipoic acid, 0.15 g, was dissolved in 25 mL of ethanol and stirred well for 5 minutes. Sodium borohydride (37 mg) was added to the solution by portions over a 2 hour period. Next, in order to derive the crude product, the ethanol was evaporated under reduced pressure, followed by treatment with saturated ammonium chloride (15 mL). The organic compound in the aqueous phase was extracted with dichloromethane (2×50 mL) and evaporated to dryness under reduced pressure. The resultant dimercapto-derivative was purified on column chromatography to get the compound in a purified form.

To confirm the presence of the dithiol compound, the dithiol compound was tested by reacting it with nitric oxide gas as the characteristic feature of thiol compounds includes the formation of a nitroso-derivative upon reaction with nitric oxide gas, as shown below.

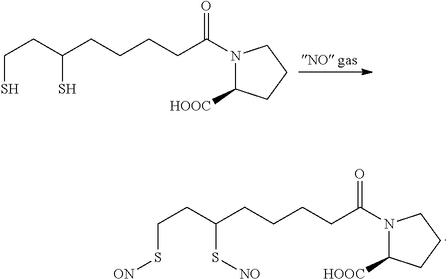

To perform this analysis, the intermediate was dissolved in ethanol (25 mL) and cooled to −20° C. using dry ice and acetone. Simultaneously, nitric oxide gas was generated by reacting sodium nitrite and concentrated hydrochloric acid. The nitric oxide gas was then passed through the dithiol solution for two hours. The solution turned a deep pink color which is characteristic of an S-nitroso compound. UV-Visible spectrum analysis of this deep pink solution further showed two characteristic absorption peaks at 330 and 540 nm, confirming the presence of the dithiol compound, which was determined to have the following chemical structure.

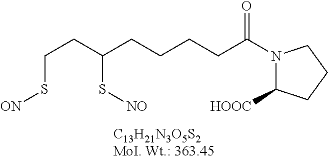

Throughout this document, various references are mentioned. All such references are incorporated herein by reference. It will also be understood that various details of the present invention can be changed without departing from the scope of the subject matter disclosed herein. Furthermore, the foregoing description is for the purpose of illustration only, and not for the purpose of limitation.

What is claimed is:

1. A compound having the Formula (I): 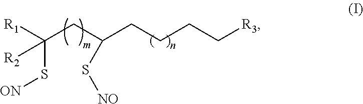
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 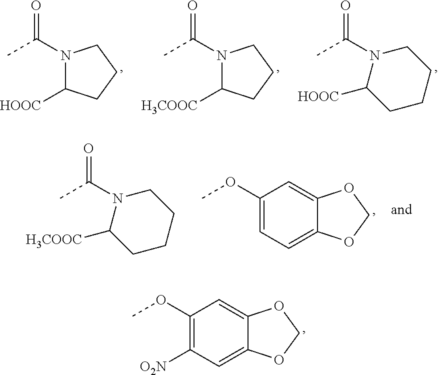
or a pharmaceutically acceptable salt or solvate thereof.

2. The compound of claim 1, wherein the compound has a formula selected from the group consisting of Formula (II):

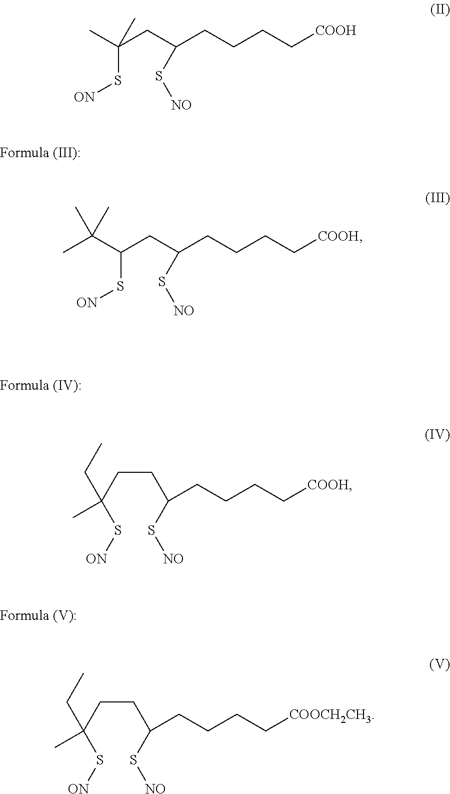

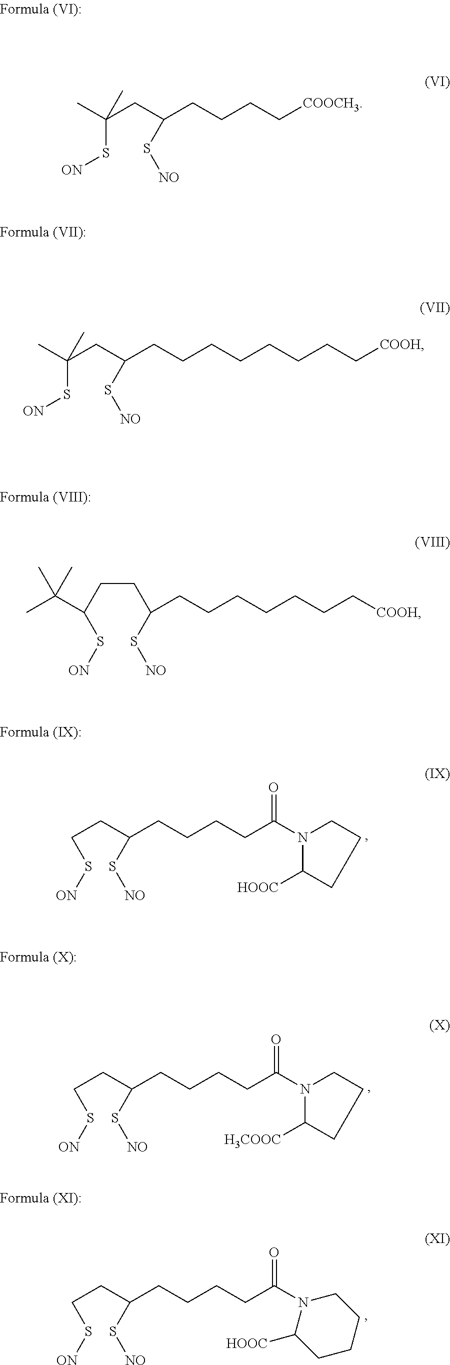

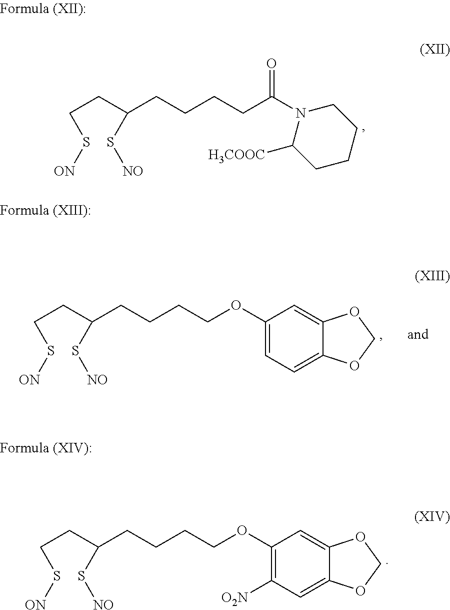

3-14. (canceled)

15. A pharmaceutical composition, comprising a compound of claim 1 and a pharmaceutically-acceptable vehicle, carrier, or excipient.

16. A compound having the Formula (XV): 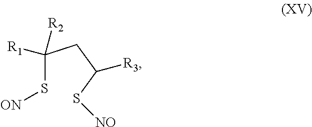
wherein:
R₁ and R₂ are independently selected from the group consisting of H, CH₃, and tert-butyl; and
R₃ is selected from the group consisting of CH₂CHCHCH₂COOCH₃, CH₂CHCHCHCHCOOH, and CHCHCHCHCOOCH₂CH₃;
or a pharmaceutically-acceptable salt or solvate thereof.

17. The compound of claim 16, wherein the compound has the Formula (XVI):

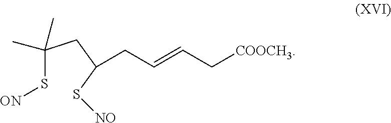

18. The compound of claim 16, wherein the compound has the Formula (XVII):

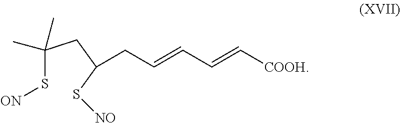

19. The compound of claim 16, wherein the compound has the Formula (XVIII):

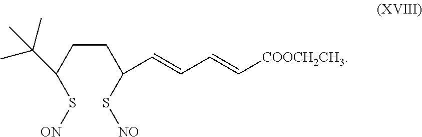

20. A method for increasing vasodilation, comprising administering to a subject in need thereof an effective amount of a compound selected from the group consisting of the following Formulas (I) and (XV), or pharmaceutically acceptable salts or solvates thereof: 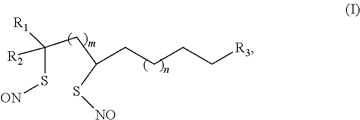
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 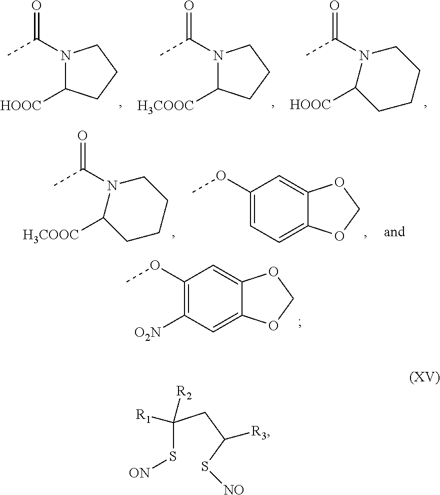
wherein:
R₁ and R₂ are independently selected from the group consisting of H, CH₃, and tert-butyl; and
R₃ is selected from the group consisting of CH₂CHCH₂CH₂COOCH₃, CH₂CHCHCHCHCOOH, and CHCHCHCHCOOCH₂CH₃.

21. A method for reducing low-density lipoprotein oxidation, comprising administering to a subject in need thereof an effective amount of a compound selected from the group consisting of the following Formulas (I) and (XV), or pharmaceutically acceptable salts or solvates thereof: 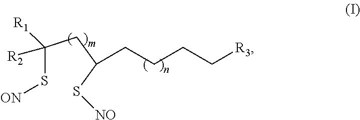
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 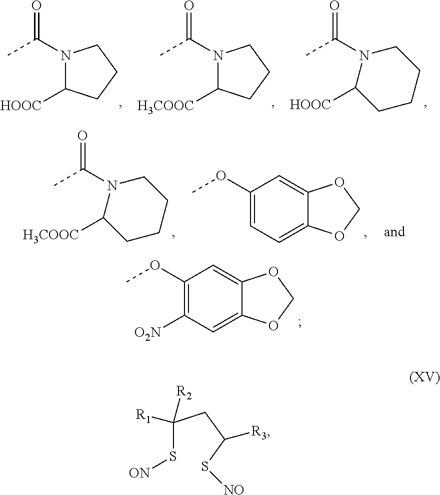
wherein:
R₁ and R₂ are independently selected from the group consisting of H, CH₃, and tert-butyl; and
R₃ is selected from the group consisting of CH₂CHCH₂CH₂COOCH₃, CH₂CHCHCHCHCOOH, and CHCHCHCHCOOCH₂CH₃.

22. A method for reducing inflammation, comprising administering to a subject in need thereof an effective amount of a compound selected from the group consisting of the following Formulas (I) and (XV), or pharmaceutically acceptable salts or solvates thereof: 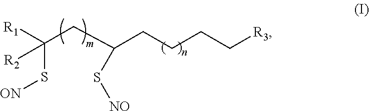
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 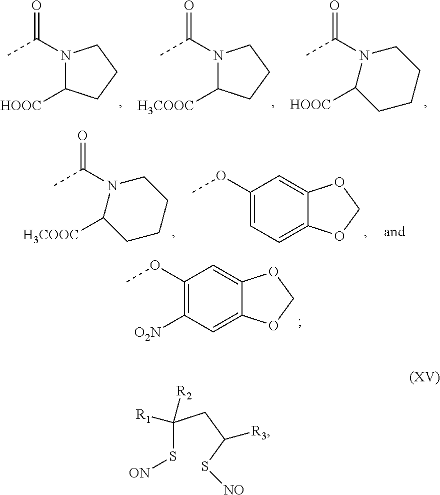
wherein:
R₁ and R₂ are independently selected from the group consisting of H, CH₃, and tert-butyl; and
R₃ is selected from the group consisting of CH₂CHCH₂CH₂COOCH₃, CH₂CHCHCHCHCOOH, and CHCHCHCHCOOCH₂CH₃.

23. A method for treating hypertension, comprising administering to a subject in need thereof an effective amount of a compound selected from the group consisting of the following Formulas (I) and (XV), or pharmaceutically acceptable salts or solvates thereof: 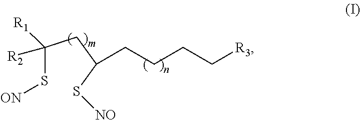
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 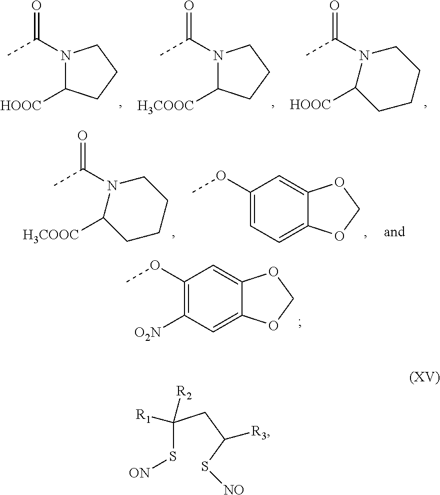
wherein:
R₁ and R₂ are independently selected from the group consisting of H, CH₃, and tert-butyl; and
R₃ is selected from the group consisting of CH₂CHCH₂CH₂COOCH₃, CH₂CHCHCHCHCOOH, and CHCHCHCHCOOCH₂CH₃.

24. A method for treating dyslipidemia, comprising administering to a subject in need thereof an effective amount of a compound selected from the group consisting of the following Formulas (I) and (XV), or pharmaceutically acceptable salts or solvates thereof: 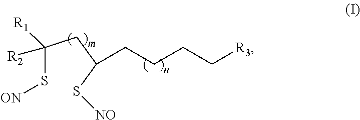
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 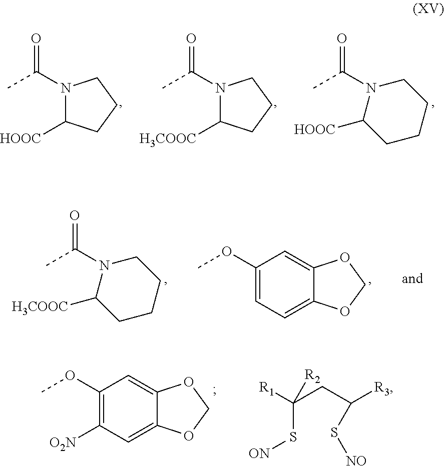
wherein:
R₁ and R₂ are independently selected from the group consisting of H, CH₃, and tert-butyl; and
R₃ is selected from the group consisting of CH₂CHCH₂CH₂COOCH₃, CH₂CHCHCHCHCOOH, and CHCHCHCHCOOCH₂CH₃.

25. A method for improving renal insufficiency or slowing the deterioration of kidney function comprising administering to a subject in need thereof an effective amount of a compound selected from the group consisting of the following Formulas (I) and (XV), or pharmaceutically acceptable salts or solvates thereof: 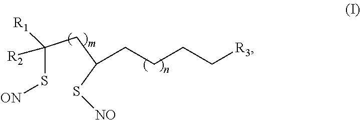
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 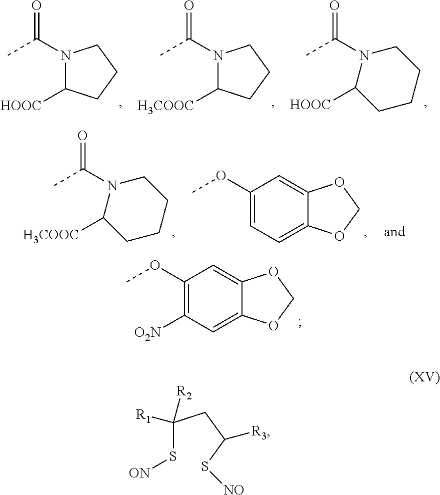
wherein:
R₁ and R₂ are independently selected from the group consisting of H, CH₃, and tert-butyl; and
R₃ is selected from the group consisting of CH₂CHCH₂CH₂COOCH₃, CH₂CHCHCHCHCOOH, and CHCHCHCHCOOCH₂CH₃.

26. The method of claim 25 wherein the method is administered to improve a conditions selected from the group consisting of diabetes or hypertension.

27. A method of reducing or preventing the progression of microalbuminuria in a subject in need thereof comprising administering to a subject in need thereof an effective amount of a compound selected from the group consisting of the following Formulas (I) and (XV), or pharmaceutically acceptable salts or solvates thereof: 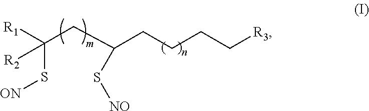
wherein:
m is an integer from 1 to 2;
n is an integer from 1 to 10;
R₁ and R₂ are independently selected from the group consisting of H, methyl, ethyl, propyl, butyl, isopropyl, isobutyl, and tert-butyl; and
R₃ is selected from the group consisting of COOH, COOCH₃, COOCH₂CH₃, 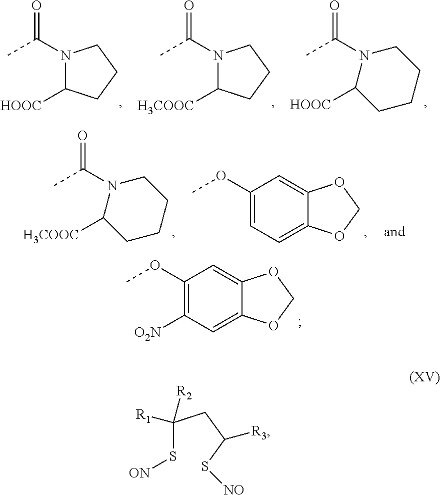
wherein:
R₁ and R₂ are independently selected from the group consisting of H, CH₃, and tert-butyl; and
R₃ is selected from the group consisting of CH₂CHCH₂CH₂COOCH₃, CH₂CHCHCHCHCOOH, and CHCHCHCHCOOCH₂CH₃.

28. A method of making a compound of claim 1, comprising:
providing alpha lipoic acid or a derivative thereof;
reducing the alpha lipoic acid or the derivative thereof to form dihydrolipoic acid or a dihydrolipoic acid derivative;
exposing the dihydrolipoic acid or derivative thereof to nitric oxide for a time sufficient to create a nitroso-form of dihydrolipoic acid; and
purifying the nitroso-form of dihydrolipoic acid.
